(19) United States
(12) Patent Application Publication
(10) Pub. No.: US 2012/0157647 A1
(43) Pub. Date: Jun. 21, 2012

(54) Polymeric Compositions and Polymerization Initiators Using Photo-Peroxidation Process

(75) Inventor: Olga Khabashesku, Houston, TX (US)

(73) Assignee: Fina Technology, Inc., Houston, TX (US)

(21) Appl. No.: 13/397,051

(22) Filed: Feb. 15, 2012

Related U.S. Application Data
(63) Continuation of application No. 12/345,525, filed on Dec. 29, 2008.

(51) Int. Cl. C08F 4/36; C07C 409/14; C07C 409/18; C07C 409/04

(52) U.S. Cl. 526/230; 568/569; 568/570; 568/567; 568/571

(57) ABSTRACT

A rubber-modified polymeric composition having predominately core-shell morphology is disclosed. The rubber-modified polymeric composition can be a polystyrene comprising styrene, polybutadiene, and a high-grafting initiator formed by contacting singlet oxygen with an olefin containing an allylic hydrogen or a diene to form a hydroperoxide or peroxide. The singlet oxygen can be formed by contacting ground state oxygen with a photo catalyst, such a photosensitive dye exposed to light.

### CROSS-REFERENCE TO RELATED APPLICATIONS

Not applicable.

### FIELD

The present invention generally relates to the production of polystyrene-polybutadiene copolymers.

### BACKGROUND

Polystyrene (PS) is a plastic made from the polymerization of the monomer styrene and is typically hard and brittle in its crystalline state. It can be made to possess certain elastomeric properties by including in its polymerization an amount of rubber, such as polybutadiene. Polystyrene that has been polymerized with an amount of rubber is termed high impact polystyrene, or HIPS. Polybutadiene is made from the polymerization of 1,3 butadiene and has unsaturated carbon-carbon double bonds in its chain that may serve as grafting sites for chains of polystyrene. Thus, when polymerized together, styrene and polybutadiene can form a graft copolymer.

The addition of polybutadiene can increase the polymer's toughness and impact absorption. HIPS can be used in a variety of applications such as casing for appliances, toys, and food containers, which require a plastic high in both gloss and impact absorption.

However, there can be an inherent trade-off between gloss and toughness in compositions of HIPS. Gloss is generally associated with polymer strength, or the polymer's hardness, a harder PS will generally have a high gloss. Toughness is related to the polymer's ability to absorb energy, a tougher PS can absorb energy and will generally have a lower gloss. A polymer high in strength is harder and less able to withstand a high energy impact than is a polymer that is softer or more rubbery.

Strength and toughness of HIPS may be influenced by several factors, including rubber particle size and morphology. For instance, large rubber particles will tend to increase the toughness of HIPS, while small rubber particles may increase hardness and gloss. The extent of grafting between the polystyrene matrix and the polybutadiene chains influences morphology. Lower levels of grafting can result in cellular or salami morphology, which is characterized by cells of rubber dispersed in the polystyrene matrix wherein each rubber cell has multiple occlusions of polystyrene either partly or completely trapped within the rubber cell. This type of morphology is generally associated with lower gloss.

A high level of grafting can lead to a core-shell morphology, in which a single polystyrene core is occluded in a polybutadiene shell and the polybutadiene shells are dispersed throughout the polystyrene matrix. Core-shell morphology is generally associated with high gloss, and is also known for achieving high transparency. It may be a suitable morphology for achieving a good balance between gloss and impact strength. Core-shell morphology also may offer an economic advantage in that a larger effective rubber particle size may be achieved with the use of less polybutadiene. Polybutadiene rubber is a relatively expensive component used in the production of HIPS. By trapping polystyrene occlusions in a rubber shell, the shell size can be expanded, as a balloon that is expanded by filling it with air.

HIPS with core-shell morphology can be difficult to obtain because of the high level of grafting required. Various methods can be employed such as the use of emulsion polymerization wherein the monomers are polymerized in a water solution with surfactant. The large amount of surfactant required, however, is a major drawback, as it may be difficult to remove after polymerization. Another method for producing HIPS can involve the use of styrene-butadiene (SBR) block copolymers instead of polybutadiene. SBR may generate a higher level of grafting than butadiene but is more expensive. Polybutadiene, though less expensive, tends to produce cellular morphology in its graft copolymer particles. Thus, an economical method of creating HIPS with a high level of grafting and core-shell morphology is desired. It would be further desirable to optimize both the economics and ecological impact of such a production method by the optional use of environmentally friendly and/or biorenewable chemicals.

### SUMMARY

Embodiments of the present invention generally include rubber-modified polymeric compositions, such as high-impact polystyrene with predominately core-shell morphology. The rubber-modified polymeric composition may comprise a matrix phase of an aromatic monomer, such as styrene, and a grafted rubber copolymer such as a polybutadiene. A high-grafting polymerization initiator can be used for grafting of the aromatic monomer to the rubber comonomer. The initiator can be formed via the reaction of singlet oxygen with an olefin containing either a diene or an allylic hydrogen, or both. Either a Diels-Alder or “ene” reaction may occur between the olefin and singlet oxygen to produce a peroxide or hydroperoxide. Peroxides and hydroperoxides are known in the art as useful initiators of vinyl polymerization, for example the mechanism by which styrene grafts to polybutadiene chains.

The olefins used as precursors of high-grafting initiators may be petrochemically derived or derived from a biorenewable source. Petrochemically derived olefins include 1,3 cyclohexadiene, 1-methyl-1-cyclohexadiene, indene, and dimethyl-2,4,6-octacyclotriene. Biorenewable olefins include alpha-terpinene, citronellol, myrcene, limonene, 3-carene, alpha-pinene, soybean oil, and farnesene.

Singlet oxygen can be formed by contacting ground-state oxygen with an activated donor, such as a photo catalyst. A photosensitive dye may form a photo catalyst upon exposure to light with a wavelength of from 300 nm to 1400 nm. Useful dyes include xanthene dye, thiazine dye, acridine dye, or combinations thereof. The dye may be sprayed onto a solid support, such as silica or alumina beads, and housed in a dry column, through which ground-state oxygen may pass. The column may be transparent, such that a light source may activate the dye, which in turn may cause the ground-state oxygen to form singlet oxygen. The dry column may be connected to a reactor, such that singlet oxygen formed in the column may pass into the reactor. The reactor may contain styrene, polybutadiene, and a high-grafting precursor olefin. Upon entering the reactor, the singlet oxygen may react with the olefin and the polybutadiene to form hydroperoxides and peroxides. These in-situ formed initiators may then be used to polymerize high impact polystyrene with a conventional temperature profile.

High-impact polystyrene may also be formed without the use of additional olefins. Polybutadiene, such as 1,4-cis-polybutadiene, may be used as a high-grafting initiator. Singlet oxygen may react with polybutadiene to form hydroperoxide groups along the polybutadiene chains. The hydroperoxide groups may serve as grafting sites for styrene, to produce a high-impact polystyrene with core-shell morphology.

The present invention can further include a method for making a rubber-modified polymeric composition comprising preparing a polymerizable mixture comprising monovinyl aromatic monomer, rubber copolymer, and a high-grafting initiator and polymerizing the mixture under reaction conditions. The high-grafting initiator is formed by contacting ground-state oxygen with an activated donor to produce singlet oxygen and contacting said singlet oxygen with an olefin containing either an allylic hydrogen or a diene, such that the olefin forms a high-grafting peroxide initiator. The high-grafting initiator facilitates grafting of monovinyl aromatic polymer along the rubber copolymer chain.

The rubber-modified polymeric composition can exhibit predominately core-shell morphology. The monovinyl aromatic monomer can be styrene or a substituted styrene compound. The grafted rubber polymer can be polybutadiene or a polymer of a conjugated 1,3-diene. The rubber-modified polymeric composition can be a high-impact polystyrene. The activated donor molecule can be obtained by exposing a photosensitive dye to light with a wavelength of from 300 nm to 1400 nm. The photosensitive dye may be selected from the following: xanthene dye, thiazine dye, acridine dye, or combinations thereof. The activated donor may be housed in a transparent dry column, through which oxygen may be passed, to form singlet oxygen.

Embodiments of the present invention include articles made from the rubber-modified polymeric compositions described herein, or made from the methods described herein.

### BRIEF DESCRIPTION OF DRAWINGS

FIG. 1a-b illustrates two examples of reactions that may occur between singlet oxygen and hydrocarbons with one or more carbon-carbon double bonds.

FIG. 2 illustrates a scheme for a laboratory reactor “dry column.”

FIG. 3 illustrates conversion, in percent solids, plotted against reaction time, in minutes, for four polymerizations. One is a control, and the other three were obtained from reactions carried out in the third example provided in the detailed description.

FIG. 4 illustrates conversion, in percent solids, plotted against reaction time, in minutes, for five polymerization involving photoperoxidized biorenewable precursors.

FIG. 5 is a TEM image of HIPS obtained with peroxidized cyclohexadiene as initiator.

### DETAILED DESCRIPTION

Embodiments of the present invention include rubber-modified polymeric compositions having predominately core-shell morphology. The rubber-modified polymeric composition may comprise a matrix phase of an aromatic monomer, such as styrene, and a grafted rubber copolymer such as a polybutadiene. A high-grafting polymerization initiator can be used for grafting of the aromatic monomer to the rubber comonomer.

The term “high-grafting” as used herein refers to a polymerization of a rubber-modified polymeric composition wherein at least 30% of the rubber chains have at least one polymer chain grafted. A high grafting initiator is one that is effective in initiating a polymerization reaction wherein at least 30% of the rubber chains have at least one polymer chain grafted

The present invention can further include a method for making a rubber-modified polymeric composition comprising preparing a polymerizable mixture comprising monovinyl aromatic monomer, rubber copolymer, and a high-grafting initiator and polymerizing the mixture under reaction conditions. The high-grafting initiator can be formed by contacting ground-state oxygen with an activated donor to produce singlet oxygen and contacting said singlet oxygen with an olefin containing either an allylic hydrogen or a diene, such that the olefin forms a high-grafting peroxide initiator. The high-grafting initiator facilitates grafting of monovinyl aromatic polymer along the rubber copolymer chain.

The present invention includes a high impact polystyrene (HIPS) with a core-shell morphology that is produced via the use of high-grafting polymerization initiators. The initiators can be formed via peroxidation by singlet oxygen.

Singlet oxygen is a reactive molecule that may be used to functionalize a variety of molecules. Singlet oxygen is a less common form of oxygen than ground-state oxygen. Ground-state oxygen is in the triplet state (indicated by the superscripted “3” in ³O₂). The two unpaired electrons in ground state oxygen have parallel spins, a characteristic that, according to the rules of physical chemistry, does not allow them to react with most molecules. Thus, ground-state or triplet oxygen is not very reactive. However, triplet oxygen can be activated by the addition of energy, causing its unpaired electrons to have opposite spins. In this way, triplet oxygen can be transformed into a reactive oxygen species, for example singlet oxygen (indicated by the superscripted “1” in ¹O₂).

.O—O. triplet oxygen (↑↑) (ground state)

↓energy

O—O: singlet oxygen (↑↓) (highly reactive)

This reaction can also be written in this form: ³O₂+energy→¹O₂*

Singlet oxygen can transfer its energy to another molecule in order to return to a low energy triplet state and is therefore useful for functionalizing a variety of molecules. For instance, hydrocarbons possessing one or more double bonds may react with singlet oxygen to form peroxides and hydroperoxides. It is well known in the art that peroxides and hydroperoxides are useful as initiators of vinyl polymerization, the type of reaction responsible for the polymerization of styrene to polystyrene and for grafting to occur between styrene and polybutadiene. Singlet oxygen may therefore be used to generate high-grafting vinyl polymerization initiators for the production of HIPS.

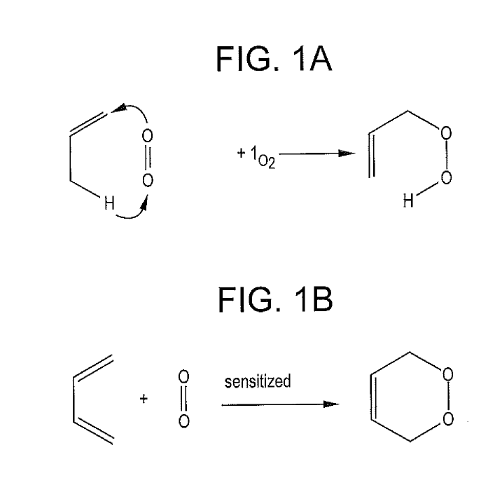

FIG. 1a-b shows two examples of reactions that may occur between singlet oxygen and hydrocarbons with one or more carbon-carbon double bonds. FIG. 1a shows an example of an “ene” reaction between singlet oxygen and a double bond system containing at least one allylic hydrogen atom. The singlet oxygen abstracts an allylic proton, and the original double bond is shifted to the allylic position, generating an allyl hydroperoxide that can act as a peroxide type initiator upon thermal decomposition. This is the type of reaction that occurs when polybutadiene is reacted with singlet oxygen. FIG. 1b shows an example of a Diels-Alder reaction between singlet oxygen and a conjugated diene. A Diels-Alder reaction generally occurs between a dienophile and a cis 1,3 diene system to create a product with two new single bonds and two less double bonds. The driving force of the reaction is the formation of new r-bonds, which are energetically more stable than π-bonds. In this case, the dienophile is singlet oxygen; it is added to a cis 1,3 diene system to create an endoperoxide. This reaction is a 1,4 cyclo addition, which has virtually zero activation energy and has a higher rate than “ene” hydroperoxidation.

The reactions shown in FIG. 1a-b both generate products that may serve as vinyl polymerization initiators. Singlet oxygen mediated additions to olefins are highly selective. No other oxygen containing derivatives are formed in these reactions. Furthermore, the reaction between singlet oxygen and olefins is of a quantitative nature, such that the amount of initiator produced may be controlled, and in turn, the level of grafting may also be controlled.

High-grafting vinyl polymerization initiators may be formed from a variety of mono- or poly-unsaturated hydrocarbons, which may undergo reactions with singlet oxygen to form hydroperoxide or endoperoxide. Some useful hydrocarbons include dienes capable of Diels-Alders reactions and olefins possessing at least one allylic hydrogen atom. Some non-limiting examples include 1,3 cyclohexadiene, 1-methyl-1-cyclohexadiene, indene, and dimethyl-2,4,6-octacyclotriene. Olefins obtained from renewable sources may also be used, including alpha-terpinene, citronellol, myrcene, limonene, 3-carene, alpha-pinene, soybean oil, and farnesene. Peroxidized hydrocarbons may be added to the polymerization reactor as high-grafting polymerization initiators or be formed in-situ simultaneously with peroxidation of polybutadiene dissolved in styrene. Hydrocarbon precursors may be in amounts of from 0.001% to 10% by weight or more of a polymerization feed. In embodiments hydrocarbon precursors may be in amounts of from 0.005% to 5% by weight of a polymerization feed. Polybutadiene may also serve as a high-grafting initiator without any extra initiators or initiator precursors. Generally, polybutadiene chains are vinyl, trans, cis, or some combination thereof A mixture of polybutadienes may be used as a high-grafting initiator. In embodiments the polybutadiene mixture may be predominately 1,4-cis-polybutadiene. The amount of polybutadiene used may range from 0.1 wt % to 50 wt % or more, or from 1% to 30% by weight of the rubber-styrene solution. If polybutadienes are added for alteration of physical properties, the amount of polybutadiene can be greater than 50 wt % of the rubber-styrene solution.

Biorenewable olefins and dienes may be produced by steam distillation of plant and seed oils. For instance, limonene may be produced from orange peel; orange peel oil is typically about 90% limonene. Pinene and myrcene may be produced from mastic gum; mastic is an evergreen shrub or small tree of the pistacio family. Myrcene is a triene olefin, which means it can serve as a bifunctional initiator with both peroxide and hydroperoxide moieties that decompose at different temperatures and act as a mixtures of initiators. Citronellol may be produced from citronella grass (lemon grass). Terpinene, a structural analog of cyclohexadiene, may be produced from cumin seeds and other plant sources. The biorenewable olefins may have the collective advantage of reducing production costs. The other unsaturated hydrocarbons that have been listed as useful largely come from petrochemical sources, and require complex synthesis in order to be produced. The biorenewable initiator precursors, in contrast, do not require complex synthesis and are available from inexpensive sources, many available from non-toxic commercially available liquids. Thus, the biorenewable olefins may provide both economic and environmental benefits.

Photoperoxidation is a process that is generally considered an environmentally friendly process and results in the generation of vinyl polymerization initiators from the above mentioned hydrocarbon precursors, both those that are petrochemically-derived and those from biorenewable sources. The process of photoperoxidation uses air and low loadings of organic dyes to transform oxygen in the air to singlet oxygen on the surface of dye illuminated with light. The singlet oxygen is generated by energy transfer from the photosensitive dye, which becomes an activated donor molecule by irradiation with electromagnetic radiation. The photosensitive dyes then can be termed photo catalysts. Electromagnetic radiation may comprise visible light with a wavelength of from 300 nm to 1400 nm. The luminous intensity may range from 20 to 90 ft candles. The lower limit of luminous intensity is generally determined by the economical yield while the upper limit is determined to avoid photo-bleaching of the photosensitive dye which can result in deactivation. The source of light may be ambient light, a tungsten lamp, a halogen lamp, or another similar light source. Some photosensitive dyes that may be used include xanthene dye, a thiazine dye, an acridine, or combinations thereof. Examples include but are not limited to rose bengal, thionin, acridine orange, methylene blue, and erythrosin.

The photosensitive dye can be suspended in the polymerization reactor such as by the flow of air through the process. The drawback to suspension of the dye in the polymerization reactor is that the dye may leach into the product. Another option is that the photosensitive dye may be supported on a solid support, such as silica or alumina beads. The solid support can be contained in a column, made of glass or other transparent material, such that the photo catalysts can be exposed to light for their activation. The column may be wet or dry, although a dry column may be desirable for avoiding the leaching of dye into the product. A dry column may comprise photo catalyst sprayed onto a solid support housed in a transparent column. Oxygen may be sparged through the column at a predetermined rate for a predetermined time, such that a controlled amount of singlet oxygen may be produced. This allows for control of the production of high-grafting initiators, and hence, of the level of grafting. Singlet oxygen produced in the dry column may then pass into a reaction vessel, containing styrene monomer, rubber, and optionally a hydrocarbon to be peroxidized.

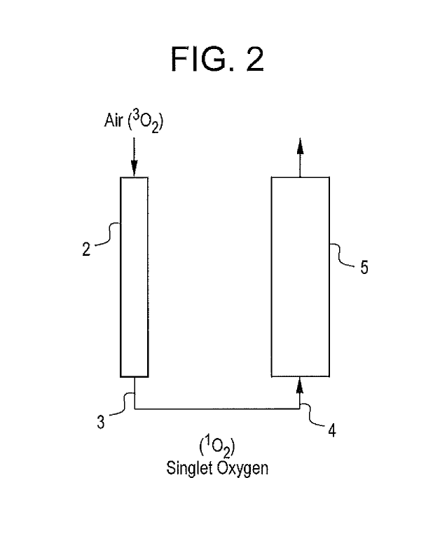

FIG. 2 shows a scheme for a laboratory reactor “dry column.” Air, which contains triplet or ground state oxygen, can be pumped through an inlet 1 into the dry column 2. The column contains silica or alumina beads or another form of solid support. The solid support has been charged with an amount of photosensitive dye. The amount of dye depends on the type of dye used, because different dyes will produce unique amounts of singlet oxygen per mol of dye per unit of light. Generally a small amount of dye, between 0.1 and 1 mg of dye per gram of support, can be used. The dry column 2 can be exposed to visible or ultraviolet light to activate the photo catalysts. As the air containing triplet oxygen passes through the column 2, the photo catalysts will transfer energy to the oxygen molecules. Thus upon exiting the column 2 through the column outlet 3, the oxygen will be singlet oxygen. The singlet oxygen will then pass into a polymerization reactor 5, through a reactor inlet 4. The contents of the reactor 5 can be mixed by the bubbling of the oxygen. The reactor 5 may comprise polybutadiene dissolved in styrene monomer. Upon reaching the reactor 5, the singlet oxygen may undergo “ene” reaction with polybutadiene to form hydroperoxide groups along the polybutadiene chain. These groups may serve as sites for high-grafting vinyl polymerization. Optionally, the reactor 5 may contain additional polyolefin initiator precursors. Upon reaching the reactor 5, singlet oxygen may react with the polyolefins to form high-grafting vinyl polymerization initiators. The reactor 5 may also contain other additives known in the art to be useful in the production of HIPS. Alternatively, the reactor 5 may contain polyolefin initiator precursors but not styrene monomer or polybutadiene. The initiator precursors may be dissolved in a solvent, and may be peroxidized within the reactor 5. Upon conclusion of the reaction, the peroxidized initiators may be drained from the reactor 5 and used in a separate reactor for HIPS polymerization.

The “dry column” process for the production of singlet oxygen offers several possible advantages, such as the use of relatively inexpensive catalysts and supports, long catalyst life, convenience of catalyst loading and removal, and no rubber deposition on the catalyst surface.

### EXAMPLES

The following examples are given as illustrative embodiments of the present invention, and are not intended to limit the scope of the invention.

In a first example, the hydroperoxidation of 1,3 cyclohexadiene was carried out in a dry column plus reactor vessel. 100 ml of 5% solution of 1,3 cyclohexadiene (Aldrich, 97%, b.p. 80° C.) in ethyl benzene was added to the laboratory photo peroxidation reactor with the dry column packed with 76 g of Rose Bengal catalyst (loading 0.26 mg/g of support) on alumina F200 (Alcoa) and sparged with air at 1 L/min for two hours. The catalyst-containing column was irradiated with a tungsten lamp (71 ft candles). After two hours, the reactor was drained, and the reaction product solution collected. Peroxide content was determined by ASTM-D-2340-82 procedure. Active oxygen was found to be 19.92 μg per ml of solution.

In a second example, hydroperoxidation reactions were run for 1-methyl-1-cyclohexadiene, indene, alpha-terpinene, and 2,6-dimethyl-2,4,6-octatriene. 100 ml of 10% solutions of each substrate (1-methyl-cyclohexadiene, Aldrich 97%, b.p. 80° C.; indene, Aldrich technical grade, b.p. 181° C.; alpha-terpinene, Aldrich 85%, b.p. 173-175° C.; 2,6-dimethyl-2,4,6-octacyclotriene, Aldrich technical grade 80%, mixture of isomers, b.p. 73-75° C./14 mm) in toluene were added to the laboratory photoperoxidation reactor with a dry column packed with 76 g of Rose Bengal catalyst (loading 0.26 mg/g of support) on silica and sparged with air at 1 L/min for two hours. Ambient lighting was used. During photo oxidation of indene, the vessel containing indene was covered to prevent light-initiated polymerization of indene.

In a third example, three hydroperoxidized rubber feeds were prepared; one in the presence of 2,3-dimethyl-2-butene, one in the presence of 1,3 cyclohexadiene, and one without any additional hydrocarbons. 170 ml of 4% solution of Diene-55 rubber in styrene monomer was added to the photoperoxidation reactor with the dry catalyst column packed with Rose Bengal supported on silica. 5 wt % of 2,3-dimethyl-2-butene was added, and the resulting mixture was sparged with air for two hours at 1 L/min flow rate. The dry column was irradiated with a tungsten lamp (71 ft candles). After two hours, the reactor was drained, and the feed was collected. A separate reaction was carried out with the addition of 5 wt % of 1,3 cyclohexadiene to the feed. At the moment of addition of the 1,3 cyclohexadiene, the feed solution noticeably thickened. A separate reaction was also carried out without the addition of any unsaturated hydrocarbon, other than rubber, as an initiator precursor.

Feeds obtained from the experiments carried out in the third example were batch polymerized using a temperature profile of 2 hours at 110° C., 1 hour at 130° C., and 1 hour at 150° C. The rates of polymerization of the photoperoxidized rubber in styrene monomer with and without the synthesized initiators appear in Table 1. These results show a significant increase in polymerization rates when the synthesized initiators are present in the photoperoxidized feed. No redox additives such a triethylamine were needed to aid thermal decomposition of these initiators.

TABLE 1
Conversion (percent solids) results of the photoperoxidations
followed by polymerizations obtained from the third example.

| | Formulation | | | |
|---|---|---|---|---|
| | | 2 | 3 | |
| | 1 | Hydro- | 2,3-dimethyl- | 4 |
| | 1,3 cyclo- | peroxidized | 2-butene | Baseline, |
| Time, | hexadiene | rubber no | added, | 170 ppm |
| min | added, 5% | additives | 5% | L-233 |
| 0 | 9.18 | 4.48 | | |
| 30 | 9.19 | 4.99 | 9.62 | |
| 60 | 11.4 | 7.88 | 12.3 | 2.41 |
| 120 | 18.52 | 15.11 | 21.5 | 9.95 |
| 180 | 38.8 | 34.05 | 43.67 | 42.97 |
| 220 | | | | 70.49 |
| 230 | | | 68.52 | |
| 240 | 78.32 | 62.08 | | |

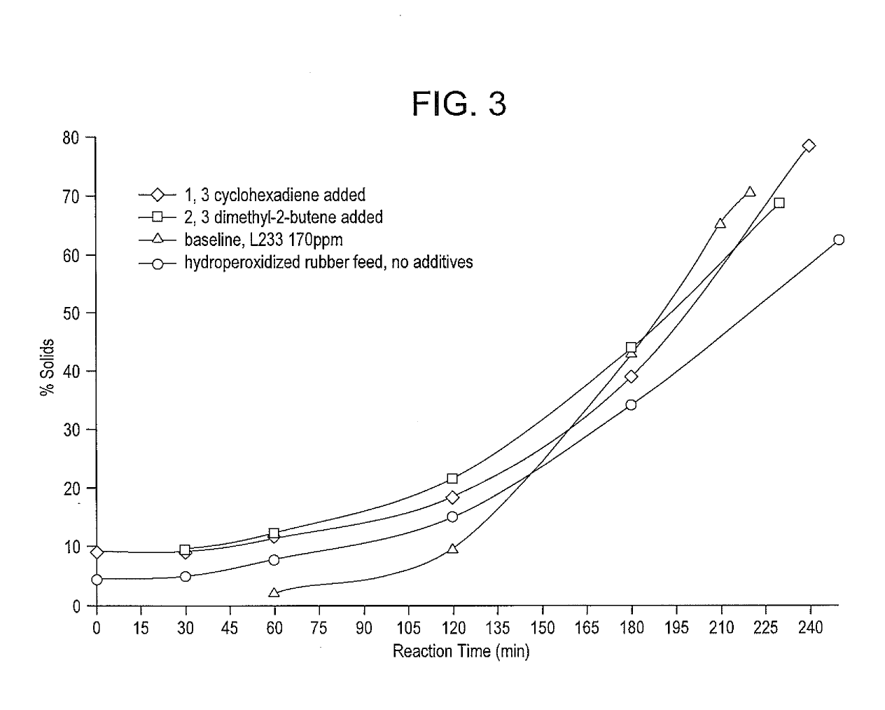

FIG. 3 shows the data from Table 1 in graph form. Conversion, in percent solids, as reaction time proceeds, is shown for four polymerizations. Line 1 corresponds to the peroxidation feed prepared with 1,3 cyclohexadiene as an initiator precursor. Line 2 corresponds to the peroxidation feed with no additional hydrocarbons as initiator precursors. Line 3 corresponds to the peroxidation feed prepared with 2,3-dimethyl-2-butene as an initiator precursor. Line 4 corresponds to a standard feed containing 170 ppm of a commercial initiator, Lupersol® L233. Photoperoxidized rubber feeds, with and without hydrocarbon initiator precursors, show polymerization rates comparable to, and in the early reaction time higher than, a feed prepared with a conventional initiator.

In a fourth example, several hydroperoxidized rubber feeds were prepared, using myrcene, limonene, alpha-terpinene, and citronellol as precursors of vinyl polymerization initiators. The olefins, purchased from Aldrich, were mixed with styrene monomer to obtain 20 wt % solutions. 100 g of each solution was photoperoxidized for two hours by singlet oxygen enriched air at 1.2 L/min airflow rate using Rose Bengal catalyst for singlet oxygen formation. Halogen light was used in addition to fluorescent light, having a light intensity between 30 and 180 ft-candles. After two hours, the peroxidized solutions were collected and 5 g of each solution were added as initiators in HIPS batch polymerizations of 200 g of 5% D55 rubber solution styrene. A standard temperature profile of 110° C. for two hours, 130° C. for one hour, and 150° C. for one hour was used.

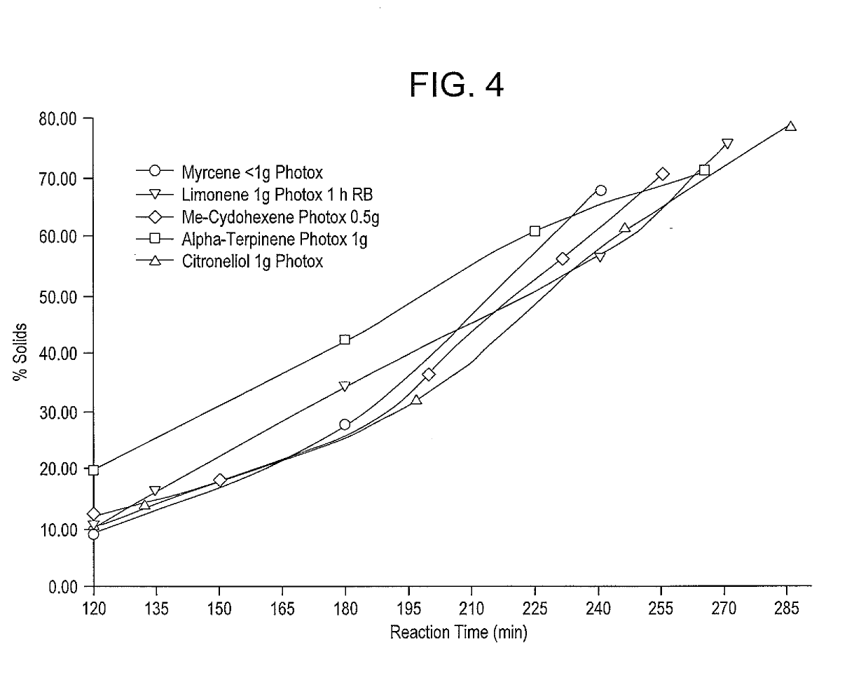

FIG. 4 is a graph showing conversion, in percent solids, as reaction time, in minutes, proceeds for the data in Table 2. Data is shown for five polymerizations using photoperoxidized biorenewable precursors. Line 1 corresponds to a feed including approximately 1 g of myrcene as the initiator. Line 2 corresponds to a feed including 1 g of limonene as the initiator. Line 3 corresponds to a feed including 0.5 g of methyl-cyclohexene as the initiator. Line 4 corresponds to a feed including 1 g of alpha-terpinene as the initiator. Line 5 corresponds to a feed including 1 g of citronellol as initiator. FIG. 4 indicates that the biorenewable compounds tested showed good polymerization activity. The rate of polymerization for this group of compounds is comparable to that of commercial initiators, such as L-233, L-531, and TMCH. Alpha-terpinene appears to be the most efficient initiator, which agrees with its highest reported rate of peroxidation by singlet oxygen.

TABLE 2

| ELAPSED | | | | | Me- |
|---|---|---|---|---|---|
| TIME | myrcene | terpinene | citronellol | limonene | cyclohexene |
| min | % solids | % solids | % solids | % solids | % solids |
| 120 | 14.63 | 11.14 | 10.10 | 10.10 | 11.14 |
| 180 | 30.10 | 25.76 | | | 25.76 |
| 195 | | | 31.19 | 31.19 | |
| 210 | | 43.79 | | | 43.79 |
| 240 | | | 57.89 | 57.89 | |
| 250 | 67.98 | | | | |
| 255 | | 70.67 | | | 70.67 |
| 265 | 73.37 | | | | |
| 285 | | | 79.14 | 79.14 | |

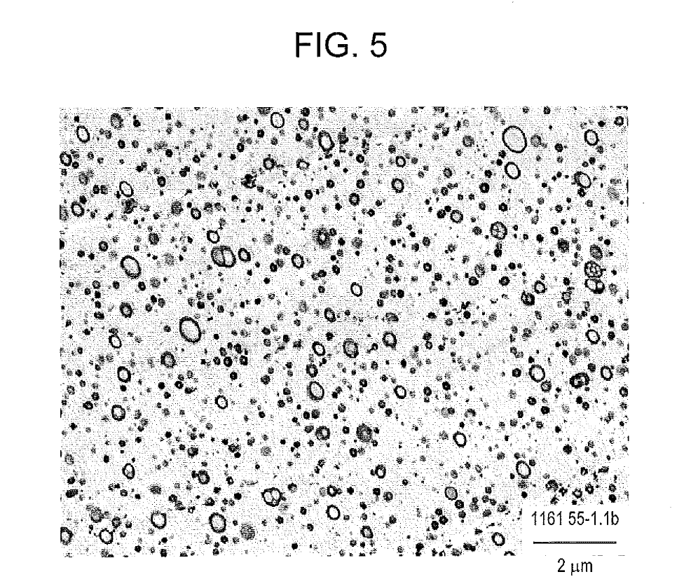

FIG. 5 shows TEM images of HIPS obtained with peroxidized cyclohexadiene as the initiator. The image shows predominately core-shell morphology, in which polystyrene cores are occluded inside polybutadiene shells, with the shells dispersed in a polystyrene matrix. This image indicates that photoperoxidation of rubber and/or other hydrocarbon initiator precursors can be used to produce HIPS with core-shell morphology.

The matrix phase of the polymer can be made from an aromatic monomer. Such monomers may include monovinylaromatic compounds such as styrene as well as alkylated styrenes wherein the alkylated styrenes are alkylated in the nucleus or side-chain. Alphamethyl styrene, t-butylstyrene, p-methylstyrene, methacrylic acid, and vinyl toluene are monomers that may be useful in forming a polymer of the invention. These monomers are disclosed in U.S. Pat. No. 7,179,873 to Reimers et al., which is incorporated by reference in its entirety.

The matrix phase of the polymer can be a styrenic polymer (e.g., polystyrene), wherein the styrenic polymer may be a homopolymer or may optionally comprise one or more comonomers. Styrene is an aromatic organic compound represented by the chemical formula C₈H₈. Styrene is widely commercially available and as used herein the term styrene includes a variety of substituted styrenes (e.g. alpha-methyl styrene), ring substituted styrenes such as p-methylstyrene, distributed styrenes such as p-t-butyl styrene as well as unsubstituted styrenes.

In an embodiment, the styrenic polymer has a melt flow as determined in accordance with ASTM D1238 of from 1.0 g/10 min to 30.0 g/10 min, alternatively from 1.5 g/10 min to 20.0 g/10 min, alternatively from 2.0 g/10 min to 15.0 g/10 min; a density as determined in accordance with ASTM D1505 of from 1.04 g/cc to 1.15 g/cc, alternatively from 1.05 g/cc to 1.10 g/cc, alternatively from 1.05 g/cc to 1.07 g/cc, a Vicat softening point as determined in accordance with ASTM D1525 of from 227° F. to 180° F., alternatively from 224° F. to 200° F., alternatively from 220° F. to 200° F.; and a tensile strength as determined in accordance with ASTM D638 of from 5800 psi to 7800 psi.

Examples of styrenic polymers suitable for use in this disclosure include without limitation CX5229 and PS535, which are polystyrenes commercially available from Total Petrochemicals USA, Inc. In a non-limiting example of an embodiment of the invention the styrenic polymer (e.g., CX5229) has generally the properties set forth in Table 3.

TABLE 3

| Physical Properties | Typical Value | Test Method |
|---|---|---|
| Melt Flow, 200/5.0 g/10 m | 3.0 | D1238 |
| Tensile Properties | | |
| Strength, psi | 7,300 | D638 |
| Modulus, psi (10⁵) | 4.3 | D638 |
| Flexular Properties | | |
| Strength, psi | 14,000 | D790 |
| Modulus, psi (10⁵) | 4.7 | D790 |
| Thermal Properties | | |
| Vicat Softening, deg. F. | 223 | D1525 |

The polymerization process may be operated under batch or continuous process conditions. In an embodiment, the polymerization reaction may be carried out using a continuous production process in a polymerization apparatus comprising a single reactor or a plurality of reactors. In an embodiment of the invention, the polymeric composition can be prepared for an upflow reactor. Reactors and conditions for the production of a polymeric composition are disclosed in U.S. Pat. No. 4,777,210, to Sosa et al., which is incorporated by reference in its entirety.

The operating conditions, including temperature ranges, can be selected in order to be consistent with the operational characteristics of the equipment used in the polymerization process. In an embodiment, polymerization temperatures range from 90° C. to 240° C. In another embodiment, polymerization temperatures range from 100° C. to 180° C. In yet another embodiment, the polymerization reaction may be carried out in a plurality of reactors, wherein each reactor is operated under an optimum temperature range. For example, the polymerization reaction may be carried out in a reactor system employing a first and second polymerization reactors that are either both continuously stirred tank reactors (CSTR) or both plug-flow reactors. In an embodiment, a polymerization reactor for the production of a styrenic copolymer of the type disclosed herein comprising a plurality of reactors wherein the first reactor (e.g., a CSTR), also known as the prepolymerization reactor, operated in the temperature range of from 90° C. to 135° C. while the second reactor (e.g., CSTR or plug flow) may be operated in the range of 100° C. to 165° C.

As used herein the term “peroxide(s)” shall include either or both of peroxide(s) and hydroperoxide(s) formed via reaction with singlet oxygen as described herein.

Use of broader terms such as comprises, includes, having, etc. should be understood to provide support for narrower terms such as consisting of, consisting essentially of, comprised substantially of, etc.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

1-28. (canceled)

29. contacting ground-state oxygen with an activated donor to produce singlet oxygen; and
contacting the singlet oxygen with an olefin containing either an allylic hydrogen or a diene to form the high-grafting peroxide initiator.

30. The method of claim 29, wherein the activated donor is obtained by exposing a photosensitive dye to light with a wavelength of from 300 nm to 1400 nm.

31. The method of claim 30, wherein the luminosity is between 20 and 90 ft candles.

32. The method of claim 31, wherein the source of the light is ambient light, a tungsten lamp, or a halogen lamp.

33. The method of claim 30, wherein the photosensitive dye is selected from the group consisting of xanthene dye, thiazine dye, acridine dye, and combinations thereof.

34. The method of claim 33, wherein the photosensitive dye is rose bengal, acridine orange, methylene blue or erythrosine.

35. The method of claim 30, wherein the high-grafting initiator is formed in a high-impact polystyrene reactor.

36. The method of claim 30, wherein the photosensitive dye is supported on a solid support.

37. The method of claim 36, wherein the support is silica or aluminum beads.

38. The method of claim 36, wherein the step of contacting ground-state oxygen with an activated donor to produce singlet oxygen further comprising passing oxygen through a transparent column containing the supported photosensitive dye to form singlet oxygen.

39. The method of claim 38, wherein the column is dry.

40. The method of claim 39 further comprising spraying the photosensitive dye onto the solid support.

41. The method of claim 29, wherein the olefin is 1,3 cyclohexadiene, 1-methyl-1-cyclohexadiene, indene, dimethyl-2,4,6-octacyclotriene, alpha-terpinene, citronellol, myrcene, limonene, 3-carene, alpha-pinene, soybean oil, or farnesene.

42. The method of claim 41, wherein the olefin is alpha-terpinene, citronellol, myrcene, limonene, 3-carene, alpha-pinene, soybean oil, or farnesene, further comprising:
forming the olefin by steam distillation of a plant oil or a seed oil.

43. The method of claim 29, wherein the oxygen includes triplet or ground state oxygen.

44. A method for forming a high-grafting initiator comprising in the presence of an olefin containing either an allylic hydrogen or a diene, contacting ground-state oxygen with an activated donor to produce singlet oxygen.
